(19) United States
(12) Patent Application Publication
(10) Pub. No.: US 2011/0124594 A1
(43) Pub. Date: May 26, 2011

(54) COMPOSITIONS THAT AIM TO PROMOTE THE DEVELOPMENT AND GROWTH OF A BENEFICIAL VAGINAL MICROFLORA

(75) Inventor: Sami Bou Antoun, Lissieu (FR)

(73) Assignee: ALLIOSPHARMA, Lissieu (FR)

(21) Appl. No.: 13/003,296

(22) PCT Filed: Jul. 9, 2009
(86) PCT No.: PCT/EP09/58756

§ 371 Date: Jan. 8, 2011

(30) Foreign Application Priority Data
Jul. 10, 2008 (FR) 0854687

(51) Int. Cl. A61K 31/715; A61P 1/00; A61P 15/02

(52) U.S. Cl. 514/54

(57) ABSTRACT

Pharmaceutical compositions and dermatological compositions comprising a prebiotic chosen among gluco-oligosaccharides (GOS), fructo-oligosaccharides (FOS), galacto-oligosaccharides and mixtures thereof; and a plant extract containing isoflavones chosen among soybean (Glycine max) extracts, clover (Trifolium sp.) extracts and mixtures thereof. Uses for the treatment of bacterial vaginoses and/or the treatment of vaginal mucosae that aim to promote the development and growth of a beneficial vaginal microflora.

The present invention relates to dermatological or pharmaceutical compositions that aim to promote the development and growth of a beneficial vaginal microflora.

In a healthy woman, urogenital flora is comprised of nearly 50 different species of microorganisms. Among these microorganisms, 95% of the population is made up of various strains of lactobacilli. These lactobacilli play a protective role against pathogens by various mechanisms including hydrogen peroxide production, lactic acid production, bacteriocin production and inhibition of pathogen adhesion and expansion. Thus, the growth of many pathogens of vaginal flora such as Gardnerella vaginalis, Prevotella bivia, Neisseria gonorrhoeae and Candida albicans is inhibited by lactobacilli. The specific lactobacilli of the vagina are notably Lactobacillus crispatus, Lactobacillus jensenii and Lactobacillus vaginalis.

Normal vaginal flora is thus principally composed of lactobacilli that form a protective biofilm on the surface of the mucosa. These lactobacilli coexist with a multitude of other species including potential pathogens. A loss of this equilibrium can be a factor that induces vaginoses due to an increase in vaginal pH and a decrease in lactobacilli, thus making it possible for pathogens to develop. These imbalances arise notably, but not exclusively, in post-menopausal women.

Antibiotic therapy is one of the treatments commonly used to treat imbalances of the vaginal microflora. It has, however, the usual disadvantages of antibiotic treatments and proves less and less effective. Moreover, it aims at eliminating pathogenic flora but it also destroys normal flora.

The products for vaginal mucosa care that already exist on the market, that aim to promote the development and growth of beneficial vaginal microflora, suffer from inadequate effectiveness.

The present invention shows in an unexpected way that the combination of a prebiotic oligosaccharide chosen among gluco-oligosaccharides (GOS) and fructo-oligosaccharides (FOS) with a plant extract containing isoflavones chosen notably among soybean and clover extracts has a synergistic effect on the growth of beneficial lactobacilli of the vaginal flora. GOS themselves have a prebiotic effect on lactobacilli growth. This result is expected and has already been described in the state of the art. Surprisingly, however, this prebiotic effect is improved in the presence of a plant extract containing isoflavones. Thus, clover extract increases lactobacilli growth and glucose consumption. This result is unexpected because plant extracts containing isoflavones are known for hormone-like effects and are used to compensate for estrogen deficits notably in post-menopausal women. A direct effect of these plant extracts containing isoflavones on lactobacilli growth had never before been documented.

The document Database WPI XP-002514149 describes compositions that include FOS/GOS and soybean extracts for the prevention of osteoporosis in women. This document does not describe a use of these compositions for the treatment and prevention of the effects of menopause on vaginal mucosa or for the treatment and prevention of bacterial vaginoses. In addition, this document does not describe compositions containing clover extracts.

FR 2 874 825 describes the use of prebiotic oligosaccharides beneficial to vaginal flora. Prebiotic oligosaccharides are chosen for their ability to promote the growth of beneficial endogenous vaginal bacterial strains. This document does not describe compositions that include both oligosaccharides and plant extracts containing isoflavones.

WO 93/00 067 describes cosmetic compositions that contain oligosaccharides. These compositions maintain an environment favourable to the development of beneficial endogenous flora. Examples of such compositions include a liquid soap, a shampoo, a body milk, a face cream as well as a vaginal gel. However, WO 93/00 067 does not describe compositions that contain both oligosaccharides and plant extracts containing isoflavones.

The document by Rousseau et al. (Anaerobe, 11: 145-153, 2005) describes the prebiotic effects of certain oligosaccharides on vaginal lactobacilli and on pathogenic microorganisms. This document does not describe compositions comprised of plant extracts containing isoflavones.

WO 2004/084 832 describes compositions that contain FO—Ti, red clover or soybean extracts that have an estrogen-type effect. These compositions are suitable notably for the treatment of effects related to the decrease of estrogens notably during menopause. However, this document does not describe compositions that also contain GOS and FOS.

EP-A-1 348 439 describes compositions for vaginal hygiene that contain isoflavones and mimosa extract. This document does not describe compositions that also contain oligosaccharides.

The document Database WPI XP-002517089 describes a method for extracting soybean isoflavones. These isoflavones can then be used for the treatment of hot flashes and vaginoses in post-menopausal women, for the prevention of cardiovascular diseases as well as for the prevention of cancers. This document does not describe compositions that also contain oligosaccharides.

The state of the art, with regard to the use of plant extracts in vaginal compositions, thus does not indicate an improvement of the prebiotic effect of oligosaccharides when they are combined with clover or soybean extracts.

The present application demonstrates that the combination of a prebiotic oligosaccharide chosen among GOS and FOS with a plant extract containing isoflavones chosen notably among soybean and clover extracts has an increased prebiotic effect on beneficial lactobacilli of the vaginal flora.

These compositions are thus particularly appropriate for the treatment of imbalances of the vaginal flora such as imbalances that follow bacterial vaginoses, candidiases or mycoses. Such imbalances of the vaginal flora can be promoted by menopause.

Vaginoses, candidiases and mycoses are usually treated by antibiotics. Following antibiotic treatment, the compositions of the present invention help to restore beneficial vaginal microflora and to restore the physiological pH of the vagina.

The invention also relates to non-therapeutic uses of the compositions of the present invention. The compositions of the present invention can be used for daily vaginal care or hygiene in order to maintain a beneficial vaginal flora and in order to maintain the physiological pH of the vagina.

The invention relates to compositions that contain:
a prebiotic chosen among gluco-oligosaccharides (GOS), fructo-oligosaccharides (FOS), and mixtures thereof; and
a plant extract that contains at least 2, 5, 10, 15, 20, 30, 40, 50 or 100 ppm isoflavones.

The invention relates to compositions that contain:
a prebiotic chosen among gluco-oligosaccharides (GOS), fructo-oligosaccharides (FOS), and mixtures thereof; and
a plant extract that contains at least 2, 5, 10, 15, 20, 30, 40, 50 or 100 ppm formononetin, biochanin A, genistein, daidzein, genistine, daidzine and/or equol.

The invention relates to compositions that contain:
a prebiotic chosen among gluco-oligosaccharides (GOS), fructo-oligosaccharides (FOS), and mixtures thereof; and
a plant extract that contains at least 2, 5, 10, 15, 20, 30, 40, 50 or 100 ppm daidzein, genistein and/or formononetin.

The invention thus relates to compositions that contain:
a prebiotic chosen among gluco-oligosaccharides (GOS), fructo-oligosaccharides (FOS), and mixtures thereof; and
a plant extract containing isoflavones chosen among soybean (Glycine max) extracts, clover (Trifolium sp.) extracts and mixtures thereof.

The invention relates more particularly to compositions for the prevention and treatment of imbalances of the vaginal microflora.

The invention relates more particularly to compositions for the prevention and treatment of bacterial vaginoses, vaginal candidiases and vaginal mycoses.

Preferably, these compositions include at least 0.5%, 1%, 2%, 3%, 4%, 5%, 6%, 7%, 8%, 9% or 10% prebiotic and at least 0.5%, 1%, 2%, 3%, 4%, 5% or 10% plant extract containing isoflavones.

Preferably, these compositions include at least 0.5% to 7% prebiotic and 0.5% to 5% plant extract containing isoflavones

Advantageously, the isoflavones are chosen among formononetin, biochanin A, genistein, daidzein, genistine, daidzine, equol and mixtures thereof.

In an advantageous embodiment, the plant extract is chosen among soybean (Glycine max) extracts, clover (Trifolium sp.) extracts and mixtures thereof.

Preferably, the plant extract is a clover extract chosen among the varieties Trifolium subterraneum L., Trifolium repens L. and Trifolium pratense L.

More preferentially, the plant extract is an extract of Trifolium pratense L. containing at least genistein, daidzein and formononetin.

In one embodiment of the invention, the prebiotic comprises an α-1,6/α-1,4 GOS having a degree of polymerization of at least 4.

In another embodiment, the prebiotic includes a FOS comprised of a mixture of FOS having a degree of polymerization of 4, 5 and 6.

The composition can be in the form of a vaginal gel, a soap, a spray or a cream for vaginal application.

The invention further relates to the use of the compositions for vaginal hygiene and care. In particular, the invention relates to the non-therapeutic use of a composition comprising a prebiotic and a plant extract containing isoflavones for vaginal hygiene and care in topical vaginal application.

The invention further relates to the non-therapeutic use of these compositions to maintain a beneficial vaginal microflora and/or to maintain vaginal pH at a physiological pH of roughly 4.5.

The invention thus relates to dermatological or pharmaceutical compositions that contain:
a prebiotic chosen among gluco-oligosaccharides (GOS), fructo-oligosaccharides (FOS), and mixtures thereof; and
a plant extract containing isoflavones.

Prebiotics are generally short-chain oligosaccharides or polysaccharides made up of roughly 2 to 20 sugar units. These are indigestible compounds that exert a beneficial action on health by selectively stimulating the growth and/or metabolic activity of one or a limited number of bacteria. The prebiotics chosen in the compositions of the present invention act as a selective substrate of one or a restricted number of beneficial bacteria that reside in the vagina and that stimulate the growth of these beneficial bacteria. The prebiotics used in the compositions of the present invention more particularly target Lactobacillus crispatus, Lactobacillus jensenii, Lactobacillus gasseri, Lactobacillus acidophilus, Lactobacillus iners and Lactobacillus vaginalis.

Most commercially available prebiotics are oligosaccharides, extracts of natural plant matrices or obtained by synthesis (chemical or biotechnological). There are various types of oligosaccharides with prebiotic potential such as gluco-oligosaccharides (GOS), fructo-oligosaccharides (FOS), galacto-oligosaccharides, etc. Prebiotic oligosaccharides of the vagina are different from prebiotic oligosaccharides of the intestine. Indeed, the intestine contains many enzymes that are likely to break down oligosaccharides. An oligosaccharide must have a long and complex chemical structure to enable it to resist enzymatic attack. Conversely, in the vagina, in the absence of enzymatic constraints, prebiotic oligosaccharides have shorter and simpler structures.

FOS are short-chain fructo-oligosaccharides made up of fructose molecules and can contain a glucose molecule at one end (according to the origin of the FOS). FOS exist in a natural state in a certain number of plants such as onion, tomato, artichoke, banana, Jerusalem artichoke, asparagus, leek, wheat, rye and garlic.

FOS can be produced industrially by two different methods:
Raftilose® FOS (Orafti, Belgium) from the hydrolysis of chicory inulin. There are other producers of this type of FOS.
Actilight® FOS (Béghin Meiji, France) obtained by enzymatic synthesis.

Actilight® FOS is produced from saccharose and an enzyme, β-fructosyltransferase, according to the following mechanism:

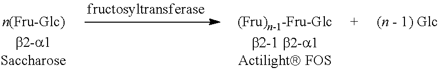

in which n is preferably equal to 3, 4 or 5.

Fructosyltransferase is produced by Aspergillus niger or Aureobasidium pullulans. From n molecules of saccharose, a mixture of saccharose, glucose and fructo-oligosaccharides is obtained. Actilight® FOS are then purified by chromatography.

Actilight® FOS contain degrees of polymerization ranging up to 6 with a mean proportion of glucose, fructose and saccharose of 5%:

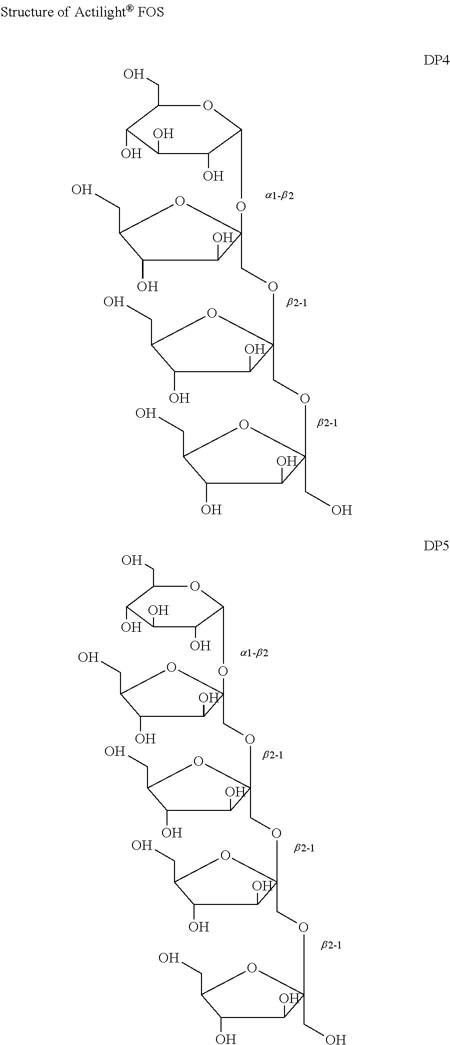

GOS are short-chain gluco-oligosaccharides made up of a chain of α1-6 linked glucose units and can also contain α1-2, α1-3 and α1-4 bonds.

α1-6 GOS are linear gluco-oligosaccharides made up of a chain of glucose units linked together by α1-6 bonds. It is obtained by an enzymatic transglucosylation reaction starting from a donor, saccharose, on an acceptor which can be glucose, methyl alpha-glucoside, isomaltose, isomaltotriose or maltose. If the acceptor is maltose (α1-4 linked glucoses) then the enzymatic synthesis of α1-6/α1-4 GOS occurs.

GOS are small glucans. They are synthesized by a transglucosylation reaction catalyzed by enzymes of the glucan-saccharase family. These enzymes ensure the transfer of glucosyl units from the donor substrate on the acceptor product at its non-reducing end. The oligosaccharide thus produced then becomes a substrate to give an oligosaccharide with a higher degree of polymerization. Glucan-saccharases accept a limited number of donor substrates (in general saccharose) but a variable number of acceptor products (for example, glucose or maltose).

There is a large variety of glucan-saccharases which have different specificities and catalyze the formation of various types of glycosidic bonds (α-(1-2), α-(1-3), α-(1-4), α-(1-6)). The yields of the reaction strongly depend on the [donor]/[acceptor] ratio and on the total sugar content in the medium. Reaction rate depends on acceptor type; for example, synthesis with maltose as acceptor is more efficient than with glucose.

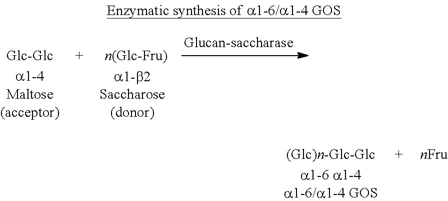

This enzymatic reaction is catalyzed by the glucan-saccharase of Leuconostoc mesenteroides NRRL B-512 (F.)

α-1,6/α-1,4 GOS are mixtures of oligosaccharides. The mixture consists of oligosaccharides ranging up to a degree of polymerization of 11.

| GOS | DONOR | ACCEPTOR | ENZYME | BONDS |
|---|---|---|---|---|
| α1-2/α1-6/ | saccharose | maltose | B-1299 | 65% α1-6 |
| α1-4 GOS | | | (1) | 35% α1-2 5% α1-3 |
| α1-6/α1-4 | saccharose | maltose | B-512F | 95% α1-6 |
| GOS | | | (2) | 5% α1-35 |

(1) glucan-saccharase of Leuconostoc mesenteroides NRRL B-1299
(2) glucan-saccharase of Leuconostoc mesenteroides NRRL B-512F
Structure of α1-6/α1-4 GOS

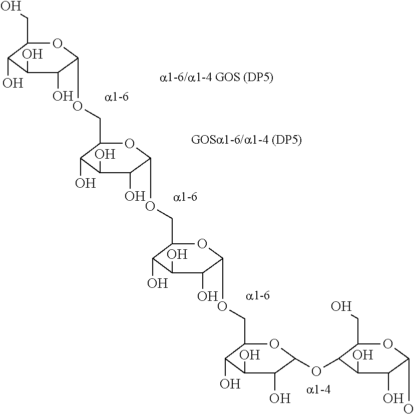

Among the prebiotics that can be used in the compositions of the present invention, the oligosaccharides described in EP-B-0 591 443, and in particular the α1-2/α1-6 GOS with a degree of polymerization of at least 4, can be cited more particularly. Another preferred prebiotic is the ActilLight® FOS of DP3.

In a preferred embodiment of the invention, the compositions contain a mixture of specific prebiotics of vaginal microflora.

The compositions of the present invention further contain plant-extract isoflavones that have a spatial structure similar to estrogens. The effects of phytoestrogens are known: they improve vaginal cytology, trophicity and hydration of vaginal mucosa, which helps make conditions for the growth of vaginal microflora more favorable. Surprisingly, these isoflavones also have a direct effect on the growth of vaginal microflora.

Among the many known sources of plant phytoestrogens, soybean (Glycine max) extracts, clover (Trifolium sp.) extracts and mixtures thereof are particularly preferred. The plant extract is typically in glycerol-coated, glycolic, aqueous or dry form or in supercritical CO₂.

These plant extracts comprise at least 2, 5, 10, 15, 20, 30, 40, 50 or 100 ppm isoflavones, preferably from 2 to 20 ppm isoflavones.

Preferably, the isoflavones are chosen among formononetin, biochanin A, genistein, daidzein, genistine, daidzine, equol and mixtures thereof. More preferentially, the plant extracts include daidzein, genistein and formononetin.

Plant extracts containing isoflavones in a sufficient quantity are prepared according to techniques that are well known to the person skilled in the art. These extracts are prepared advantageously from clover or from soybean. These extracts are available commercially and one such example is the Sterocare® clover extract sold by Sederma.

In a preferred embodiment, the compositions of the present invention include an extract of clover Trifolium pratense L. containing at least 2 ppm daidzein, at least 11 ppm genistein and at least 9 ppm formononetin.

The compositions can further include at least one gelling agent of natural plant, mineral or synthetic origin, which in contact with water forms a thixotropic hydrocolloidal gel with no electrical charge. Examples include polymer derivatives of acrylic acid, hydroxyethyl cellulose, carboxymethyl cellulose, xanthan gums, guar gums, galactomannans, succinoglycans, kappa or iota carrageenans, bentonites, hectorites and magnesium silicates. The gelling agent represents from 0.5% to 5% in the final composition

The compositions can further include a glycol such as monopropylene glycol, dipropylene glycol, glycerol, diglycerol, triglycerol, monopropanediol, butylene glycol or hexylene glycol. It can be added to the composition in a proportion of 1% to 80%, preferably a proportion of 3% to 10% of the total composition.

The compositions of the invention can further include a preservative system composed of 1 to 4 fungistatic and bacteriostatic agents, in the form of organic acid salts such as lactic, citric, sorbic, benzoic, salicylic, tartaric, glycolic, etc. They can be added to the composition in a proportion between 0.1% and 2% of the total composition.

Advantageously, the compositions will include pH adjusters to bring the preparation to physiological pH such as citric, lactic, glycolic, hydrochloric or boric acid, sodium hydroxide, potassium hydroxide, cyclic AMP, triethanolamine, tromethamine, ethylene diamine tetra acetic acid, citric/citrate buffer, etc. pH adjusters are added to the composition in a proportion between 0.05% and 5% of the final composition, particularly between 0.05% and 1% of the final composition.

The compositions can further include at least one compound chosen from the group comprised of:
active ingredients typically used in dermatology such as emollients, hydrating active ingredients, cutaneous barrier restructuring agents, anti-irritants, soothing agents,
active ingredients that have a complementary therapeutic action, such as antibiotics, antibacterials, antifungals, antivirals, anti-inflammatories, etc.

The compositions of the invention can be formulated in the form of various preparations suitable for topical, oral or vaginal administration.

According to a preferred embodiment, the various preparations are suitable for topical application and include creams, emulsions, milks, pomades, lotions, oils, aqueous or hydro-alcohol or glycol solutions, powders, sprays, jellies, gels, hydrogels or any other product for external application.

The invention further relates to pharmaceutical compositions that contain a prebiotic and a plant extract containing isoflavones as defined above as well as a suitable pharmaceutical carrier.

In the pharmaceutical compositions of the present invention for oral administration, the active ingredient can be administered in unit dosage forms, in mixture with traditional pharmaceutical supports. Suitable unit dosage forms include forms by oral route such as tablets, gelatine capsules, powders, granules and oral solutions or suspensions.

When a solid composition in tablet form is prepared, the principal active ingredient is mixed with a pharmaceutical carrier such as gelatine, starch, lactose, magnesium stearate, talc, gum arabic or analogues. Tablets can be coated with saccharose or other suitable materials or can be treated in such a way that they have extended or delayed activity and that they continuously release a predetermined quantity of active ingredient.

A preparation in gelatine capsules is obtained by mixing the active ingredient with a diluent and by pouring the mixture obtained into soft or hard gelatine capsules.

A preparation in syrup or elixir form can contain the active ingredient together with a sweetener, an antiseptic, as well as a flavouring agent and a suitable colorant.

Water-dispersible powders or granules can contain the active ingredient in mixture with dispersion or wetting agents, or suspension agents, as well as with flavor correctors or sweeteners.

Preferably, the pharmaceutical compositions of the present invention are provided in the form of a gel, a jelly, a soap, a spray or a cream for topical vaginal application.

The compositions of the invention are more particularly suitable for the treatment and prevention of imbalances of the vaginal microflora, in particular for the prevention and treatment of bacterial vaginoses, vaginal candidiases and vaginal mycoses.

The effects of menopause on the mucosae can more particularly promote the development of these imbalances of the vaginal microflora.

“Vaginosis” or “bacterial vaginosis” means an imbalance of the microbial flora of the vagina. It is characterized by the disappearance of lactobacilli and the multiplication of anaerobic bacteria such as Gardnerella vaginalis. It indicates an imbalance of the vaginal flora with the disappearance of the protective effect of Döderlein's bacillus.

“Imbalance of the vaginal microflora” means any deficit in lactobacilli and in particular a deficit in Lactobacillus crispatus, Lactobacillus jensenii, Lactobacillus gasseri, Lactobacillus acidophilus, Lactobacillus iners or Lactobacillus vaginalis.

“Vaginal candidiasis” means an imbalance of the vaginal microflora or a vaginal infection caused by yeasts of the genus Candida.

“Vaginal mycoses” means imbalances of the vaginal microflora more generally caused by fungi.

The invention further relates to methods of therapeutic treatment of imbalances of the vaginal microflora comprising the administration to an individual of an effective quantity of a composition of the invention.

The invention further relates to methods of therapeutic treatment of bacterial vaginoses, vaginal candidiases and vaginal mycoses, comprising the administration to an individual of an effective quantity of a composition of the invention.

The invention further relates to the use of one of these compositions for the manufacture of a drug for the treatment and prevention of imbalances of the vaginal microflora and more particularly for the treatment and prevention of bacterial vaginoses, vaginal candidiases and vaginal mycoses.

The invention further relates to the use of one of these compositions for the manufacture of a drug for the treatment and prevention of the effects of menopause on vaginal mucosae.

The invention further relates to the use of a prebiotic and a plant extract containing isoflavones as defined above for the manufacture of a drug for the treatment and prevention of imbalances of the vaginal microflora and more particularly for the treatment and prevention of bacterial vaginoses, vaginal candidiases and vaginal mycoses.

The invention further relates to the use of a prebiotic and a plant extract containing isoflavones as defined above for the manufacture of a drug for the treatment and prevention of the effects of menopause on vaginal mucosae.

The invention further relates to dermatological compositions for vaginal hygiene and vaginal care. The compositions of the present invention are particularly suited for the vaginal hygiene and care of post-menopausal women.

In the context of a dermatological use, the composition will advantageously be formulated in the form of a preparation suitable for topical application.

The invention also relates to methods of vaginal hygiene and vaginal care, notably for post-menopausal women.

The invention further relates to the non-therapeutic use of the compositions of the invention for vaginal care and hygiene. In particular, the invention relates to the non-therapeutic use of a composition comprising a prebiotic and a plant extract containing isoflavones for vaginal hygiene and care in topical vaginal application.

The invention further relates to the non-therapeutic use of these compositions to maintain a beneficial vaginal microflora and/or to maintain vaginal pH at a physiological pH of roughly 4.5.

### FIGURES

FIG. 1: Growth of Lactobacillus crispatus BLL 2008

FIG. 2: Growth of Lactobacillus crispatus BLL 2005

### EXAMPLES

### Example 1

### Vaginal Jelly

| RAW MATERIAL | % |
|---|---|
| Bio-osmotic water | QSP 100 |
| Aqua | |
| Plant glycerol codex | 5 |
| Glycerol | |
| αGOS | 5.0 |
| Alpha-glucan/oligosaccharide | |
| (GENIBIO ®) | |
| Sterocare ® (Sederma ®) | 2.0 |
| Trifolium Pratense extract | |
| Natrosol ® 250 HHX.Ph | 1.0 |
| Hydroxyethyl cellulose | |
| Ronkosal ® SB | 0.7 |
| Preservative | |
| Purac ® HS 90 | QSP pH 4.3 ± 0.1 |
| Lactic acid | 0.22 |

The concentration of Sterocare® can also vary from 0.5% to 3%. The rest of the formula stays unchanged. For a gel formulation, fatty agents are added.

Sterocare® comprises 2 ppm daidzein, 11 ppm genistein and 9 ppm formononetin.

### Example 2

### Placebo Gel Formula

| RAW MATERIAL | % |
|---|---|
| Bio-osmotic water | QSP 100 |
| Aqua | |
| Plant glycerol codex | 5 |
| Glycerol | |
| Natrosol ® 250 HHX.Ph | 1.0 |
| Hydroxyethyl cellulose | |
| Ronkosal ® SB | 0.7 |
| Preservative | |
| Purac ® HS 90 | QSP pH 4.3 ± 0.2 |
| Lactic acid | 0.3 |

This is the same formula without GOS and Sterocare®

### Example 3

### Washing Foam (or Personal Hygiene Gel)

| Raw material | % |
|---|---|
| Purified water | QSP 100 |
| Aqua | |
| Proteol ® APL | 15 |
| Sodium cocoyl apple amino acids | |
| Foam derived from apple | |
| Laureth P065 | 3 |
| Aqua, coco-glucoside, glyceryl oleate | |
| αGOS | 1 |
| Alpha-glucan/oligosaccharide | |
| (Genibio ®) | |
| Red clover HG | 1 |
| Trifolium pratense, glycerol, water | |
| Active ingredient | |
| (Greentech ®) | |
| Sepinov ® EMT10 | 0.7 |
| Hydroxy ethyl acrylate/sodium | |
| (stabilizer) | |
| Suttocide ® A | 0.5 |
| Sodium methyl hydroxy glycinate | |
| (preservative) | |
| Purac ® HS 90 | 0.4 (pH 5-5.5) |
| Lactic acid | |
| (pH adjuster) | |
| Mint green tea 12309 (fragrance) | 0.06 |
| Red dye EC approved | 0.00066 |
| CI 16035 | |
| EDTA | 0.05 |
| Ethylene diamine tetra acetic acid | |
| (sequestering agent) | |

The concentrations in GOS and red clover can vary between 0.5% and 1.5%.

### Example 4

### Gelatin Capsule

Prebiotic Oligosaccharides (GOS and/or FOS)

Isoflavones (of clover and/or soybean)

Excipients: lactose monohydrate, potato starch, sodium glutamate, sodium thiosulfate, gelatine capsule shell (E171 titanium dioxide, gelatine).

### Example 5

### Cream

Prebiotic oligosaccharides (GOS and/or FOS)

Isoflavones (of clover and/or soybean)

Excipients: petroleum jelly, nonionic emulsifiable wax in cetomacrogol 1000, liquid paraffin, glycine, sorbic acid, concentrated sodium hydroxide solution qsp pH 4-5, purified water.

### Example 6

We chose two strains of lactobacilli, BLL 2005 and 2008 (the two most significant strains in terms of probiotic activity), for which we tested 1) glucose alone, which is the universal substrate of lactobacilli, and glucose+Sterocare® (1.5% and 3%), and 2) GOS alone and GOS+Sterocare® (1.5% and 3%).

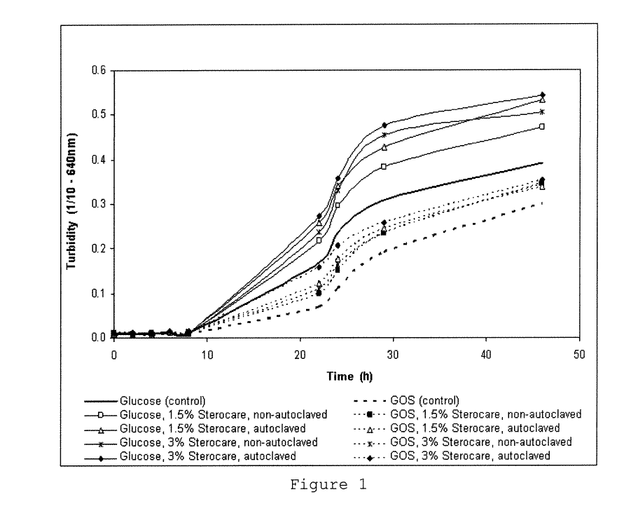

In both cases, we observe that Sterocare® increases substrate consumption by lactobacilli and thus their growth (FIG. 1).

### Example 7

### Lactic Bacteria Growth Over Time: Growth in the Presence or Absence of Vaginal Hygiene Gel

Initial sugar concentration 5 g/l; 2% inoculation.

The active-formula and placebo-formula vaginal hygiene gels of Examples 1 and 2 were used.

| | BLL 2005 - Lactobacillus crispatus | | | | | |
|---|---|---|---|---|---|---|
| | MRS + Glucose | | MRS + Placebo | | MRS + Active gel | |
| | Turbidity | | Turbidity | | Turbidity | |
| Temps | 1/10 - | | 1/10 - | | 1/10 - | |
| (h) | 640 nm | pH | 640 nm | pH | 640 nm | pH |
| Pre- | 0.328 | 4.0 | / | / | / | / |
| culture | | | | | | |
| 0.0 | 0.005 | 6.5 | 0.004 | 6.4 | 0.005 | 6.4 |
| 2.5 | 0.005 | / | 0.005 | / | 0.004 | / |
| 4.0 | 0.008 | / | 0.007 | / | 0.005 | / |
| 6.0 | 0.014 | 4.9 | 0.011 | 6.0 | 0.011 | 6.2 |
| 24.0 | 0.214 | 4.9 | 0.022 | 6.0 | 0.166 | 5.2 |
| 29.5 | 0.224 | / | 0.022 | / | 0.190 | / |
| 46.5 | 0.195 | 4.9 | 0.021 | 6.0 | 0.185 | 5.1 |

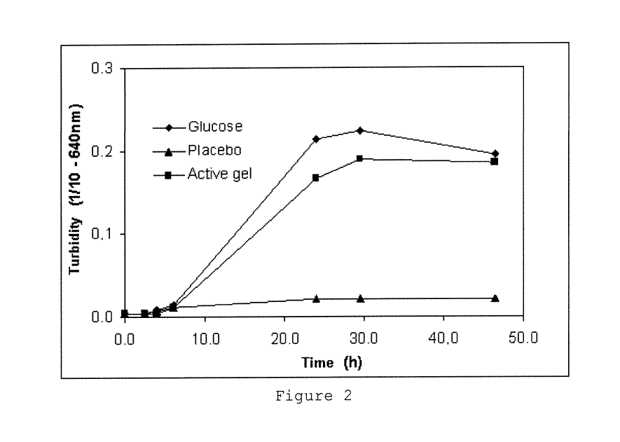

The results presented above and in FIG. 2 show without ambiguity that the active gel enables growth of the strain, which is not the case with the placebo.

### Example 8

### Comparison of Nugent Scores and Vaginal pH in Treated and Untreated Women

### Protocol

Treatment of two groups of women having had a bacterial vaginosis and treated by an antibiotic. The first group receives the active product and the second (placebo) group receives the product without prebiotics and clover extract.

The treatment consists in applying the products (the one containing prebiotics and isoflavones and the placebo) once per day. The product is provided in the form of disposable single-dose tubes containing 5% GOS and the plant extract containing 2% isoflavones (vaginal jelly Example 1).

The treatment is daily for 8 days.

At D0, the women are auscultated by a gynecologist who takes a vaginal sample and measures vaginal pH.

At D8, a second appointment and vaginal sample and pH measurement.

Thus, at D0 and D8, Nugent scores and pH are measured in order to compare scores and pH within the same group and to compare the subjects of the two groups.

### Analysis of Nugent Scores

Objective of the Nugent Score:
To determine the quality of vaginal flora
To differentiate normal vaginal flora, intermediate vaginal flora (can develop into vaginosis) and vaginoses.
   Comparison of changes in Nugent scores between the active group and the placebo Group.

| Nugent score | | |
|---|---|---|
| Normal | Intermediate | |
| range | range | Vaginosis |
| (0-3) | (4-6) | (7-10) |

| Subjects | D0 | D8 | D0 | D8 | D0 | D8 |
|---|---|---|---|---|---|---|
| Active | 72% | 100% | 28% | 0% | 0% | 0% |
| Placebo | 67% | 56% | 33% | 33% | 0% | 11% |

After 8 Days:

The active group shows after 8 days of treatment a restoration of normal vaginal flora for all subjects.

The placebo group shows a more random dispersion after 8 days with 33% of individuals characterized by intermediate flora and 11% by vaginosis.

Statistical analysis of Nugent scores at D8.

| | | Standard | |
|---|---|---|---|
| D8 Nugent score | Mean | deviation | Interval |
| Active (8) | 1 | 1 | [0-2] |
| Placebo (9) | 3 | 3.67 | [0-6.6] |

A greater variability of Nugent scores is observed in the placebo group versus the active group. The standard deviation is indeed 2.5 times higher in the placebo population.

Kinetic change in active group Nugent scores after 8 days.

| | Stabilization of | Restoration of |
|---|---|---|
| | normal vaginal | normal vaginal |
| | flora | flora |
| Active (8) | 75% | 25% |

Treatment led to a Nugent score that is stabilized below 3 after 8 days.

75% of subjects have a score≦1

12.5% of subjects have a score=2

12.5% of individuals have a score from 5 to 3.

### CONCLUSION

On the basis of this sample, the treatment has a significant effect on the active group and led to a quicker restoration of normal vaginal flora.

11% of individuals in the placebo group had a recurrence of vaginosis after 8 days.

### Analysis of Vaginal pH

The physiological pH of the vaginal mucosa is acidic and roughly 4.5.

Vaginosis is characterized by a higher pH.

Comparison of vaginal pH change between the active group and the placebo group.

| | |
|---|---|
| pH ≦ 4 | pH > 4 |

| Subjects | D0 | D8 | D0 | D8 |
|---|---|---|---|---|
| Active | 50% | 71% | 50% | 29% |
| Placebo | 55% | 33% | 45% | 67% |

After 8 Days:

The vaginal pH of subjects in the active group tends to decrease after 8 days of treatment versus the subjects in the placebo group in which an opposite effect is noted.

Variation of vaginal pH in subjects of the active group between D0 and D8.

| | A1 | A3 | A4 | A5 | A7 | A8 | A9 |
|---|---|---|---|---|---|---|---|
| ΔpH (D0-D8) | 0 | −1 | 0.5 | 0 | 2 | 2 | 0 |

Variation of vaginal pH in subjects of the placebo group between D0 and D8.

| | P1 | P2 | P3 | P4 | P5 | P6 | P7 | P8 | P9 |
|---|---|---|---|---|---|---|---|---|---|
| ΔpH (D0-D8) | −2 | 0 | 1.5 | −1 | −0.5 | 0 | 0 | 0 | 0 |

86% of the subjects in the active group show a stabilisation or a decrease in vaginal pH.

28% have a final pH equal to 5.

66% of the subjects in the placebo group show a stabilisation or a decrease in vaginal pH, knowing that in 66% of the cases this final pH is equal to or greater than 4.5.

### CONCLUSION

On the basis of this sample, the treatment has a significant effect on the active group and:
promotes a decrease in pH towards physiological values (4-4.5), or
maintains a vaginal physiological pH restored by antibiotic treatment.

Stability or decrease in vaginal pH in subjects of the placebo group reaches a physiological value in only 33% of cases.
The effect of the treatment on subjects in the active group has a significant positive effect on the quality of flora and vaginal pH.
An overall correlation is noted between the tendency to a normal flora and the tendency to an acidic pH reflecting a prevalence of “commensal” lactic bacteria.

### Example 9

### Isoflavone Assay

The objective of this study was to test the presence of prebiotics and isoflavones in the blood of doe rabbits following the topical application of a composition of the invention.

Two doe rabbits per group received once per day for 8 days a topical intra-vaginal application of control gel (petroleum jelly) and the gel of Example 1 (gel containing 5% GOS and plant extract containing 2% isoflavones). Blood samples were drawn one day before the applications (T₀) and one day after the end of the applications (T₈).

By HPLC, it was not possible to detect the presence of isoflavones (daidzein, genistein and formononetin) or GOS in the plasma of the doe rabbits of the treated group and of the control group. As a positive control, the plant extract of clover used in the compositions includes 2 ppm daidzein, 11 ppm genistein and 9 ppm formononetin.

The compositions thus have only a topical effect; the active components, GOS and isoflavones, are not absorbed and do not have a systemic effect. The effect of isoflavones is thus not a hormone-like effect.

1. A composition for the prevention and/or treatment of imbalances of the vaginal microflora comprising:
a prebiotic chosen among gluco-oligosaccharides (GOS), fructo-oligosaccharides (FOS), and mixtures thereof; and
a plant extract comprising at least one isoflavone chosen among formononetin, biochanin A, genistein, daidzein, genistine, daidzine, equol and mixtures thereof.

2. The composition of claim 1, suitable for the prevention and/or treatment of bacterial vaginoses, vaginal candidiases and vaginal mycoses.

3. The composition of claim 1, suitable for the restoration of a beneficial vaginal flora following an antibiotic treatment.

4. The composition of claim 1, suitable for the restoration of a vaginal physiological pH of roughly 4.5.

5. The composition according to claim 1, comprising 0.5% to 7% prebiotic and 0.5% to 5% plant extract comprising isoflavones.

6. The composition according to claim 1, wherein the plant extract comprises at least 10 ppm isoflavones.

7. The composition according to claim 1, wherein the plant extract is chosen among soybean (Glycine max) extracts, clover (Trifolium sp.) extracts and mixtures thereof.

8. The composition according to claim 1, wherein the plant extract is a clover extract chosen among the varieties Trifolium subterraneum L., Trifolium repens L. and Trifolium pratense L.

9. The composition according to claim 1, wherein the plant extract is an extract of Trifolium pratense L. comprising at least one of genistein, daidzein and/or formononetin.

10. The composition according to claim 1, wherein the prebiotic comprises an α-1,6/α-1,4 GOS having a degree of polymerization of at least 4.

11. The composition according to claim 1, wherein the prebiotic comprises a FOS comprised of a mixture of FOS having a degree of polymerization of 4, 5 and 6.

12. The composition according to claim 1, wherein the composition is in the form of a vaginal gel, a soap, a spray or a cream for vaginal application.

13. A method for vaginal hygiene and care comprising:
topical vaginal application to an individual of an effective quantity of a composition comprising:
a prebiotic chosen among gluco-oligosaccharides (GOS), fructo-oligosaccharides (FOS), and mixtures thereof; and
a plant extract containing at least one isoflavone chosen among formononetin, biochanin A, genistein, daidzein, genistine, daidzine, equol and mixtures thereof.

14. The method of claim 13, wherein said method permits said individual to maintain a beneficial vaginal microflora.

15. The method according to claim 13, wherein said method permits said individual to maintain vaginal pH at a physiological pH of roughly 4.5.
